(19) United States
(12) Patent Application Publication
(10) Pub. No.: US 2018/0318246 A1
(43) Pub. Date: Nov. 8, 2018

(54) PHARMACEUTICAL COMPOSITIONS OF DIMETHYL FUMARATE

(71) Applicant: SUN PHARMACEUTICAL INDUSTRIES LIMITED, Mumbai, Maharashtra (IN)

(72) Inventors: Chandrashekhar GARGOTE, Gurgaon (IN); Lalit GARG, New Delhi (IN); Shrikant Vaijanathap HODGE, Nanded (IN); Subodh DESHMUKH, Quaker Hill, CT (US); Romi Barat SINGH, Varanasi (IN); Vikas BATRA, Karnal (IN); Som Akshay JAIN, Sagar (IN)

(21) Appl. No.: 15/772,455

(22) PCT Filed: Oct. 27, 2016
(86) PCT No.: PCT/IB2016/056479

§ 371 Date: Apr. 30, 2018

(30) Foreign Application Priority Data
Oct. 28, 2015 (IN) 3504/DEL/2015

(51) Int. Cl. A61K 31/225; A61K 9/00; A61K 9/19; A61K 9/28; A61K 9/48; A61K 47/18; A61K 47/20; A61K 47/26; A61P 17/06; A61P 25/00

(52) U.S. Cl. CPC A61K 31/225; A61K 9/0053; A61K 9/19; A61K 9/2833; A61P 25/00; A61K 47/183; A61K 47/20; A61K 47/26; A61P 17/06; A61K 9/4808

(57) ABSTRACT

The present invention relates to pharmaceutical composition comprising dimethyl fumarate; an enzyme modulator or a permeation enhancer or both; and one or more pharmaceutically acceptable excipients. It further relates to a pulsatile release pharmaceutical composition comprising dimethyl fumarate and one or more pharmaceutically acceptable excipients. The compositions of the present invention are administered at a lower dose as compared to the recommended daily dose of Tecfidera®. Further, the compositions of the present invention are resistant to dose dumping in the presence of alcohol.

### FIELD OF THE INVENTION

The present invention relates to a pharmaceutical composition comprising dimethyl fumarate; an enzyme modulator or a permeation enhancer or both; and one or more pharmaceutically acceptable excipients. It further relates to a pulsatile release pharmaceutical composition comprising dimethyl fumarate and one or more pharmaceutically acceptable excipients. The compositions of the present invention are administered at a lower dose as compared to the recommended daily dose of Tecfidera®. Further, the compositions of the present invention are resistant to dose dumping in the presence of alcohol.

### BACKGROUND OF THE INVENTION

Dimethyl fumarate is the dimethyl ester of fumaric acid. Dimethyl fumarate is commercially available as Tecfidera® delayed release capsules in 120 mg and 240 mg strengths for the treatment of multiple sclerosis. The recommended therapeutic dose of Tecfidera® is 240 mg twice a day, i.e., 480 mg per day. Tecfidera® displays dose-dependent adverse effects, mainly flushing and gastrointestinal complaints such as abdominal pain, diarrhea, and nausea, which results in discontinuation of therapy in about 4% of patients. Administration with food reduces the incidence of flushing, however the Cₘₐₓ of monomethyl fumarate, an active metabolite of dimethyl fumarate, is decreased by 40% and the Tₘₐₓ is delayed from 2.0 hours to 5.5 hours. Further, as per the US FDA's Summary Basis of Approval, Tecfidera® is susceptible to alcohol-induced dose dumping and, when administered with alcohol, the entire drug is released within 30 minutes of administration leading to enhanced adverse effects and reduced efficacy of the drug.

PCT Publication No. WO 00/30622 and U.S. Pat. Nos. 6,277,882 and 6,355,676 disclose that side effects of dimethyl fumarate and/or monomethyl fumarate may be reduced by administering them in the form of microtablets.

PCT Publication No. WO 2006/037342 discloses controlled release pharmaceutical compositions comprising fumaric acid ester(s) wherein the controlled release profile results in a reduction in gastro-intestinal related side-effects.

PCT Publication Nos. WO 2010/079222, WO 2015/028472, and WO 2015/028473 disclose dimethyl fumarate formulations in the form of erosion matrix tablets with reduced gastro-intestinal related side-effects and/or reduced flushing. WO 2015/028472 discloses a dose of 400 mg±5% or 410 mg±5% per day for treating multiple sclerosis and WO 2015/028473 discloses a dose of 375 mg±5% per day for treating psoriasis.

There exists a need in the art to develop alternate low dose formulations of dimethyl fumarate having reduced adverse effects. Further, there exists a need in the art to provide a formulation of dimethyl fumarate which has reduced food effects and a reduced potential for alcohol-induced dose dumping.

After oral administration, dimethyl fumarate is extensively metabolized by esterases via hydrolysis into its active metabolite, monomethyl fumarate, before it reaches systemic circulation. The main sites of this metabolism include the gastrointestinal tract, blood, and tissues. The adverse effects associated with dimethyl fumarate may be scaled down by reducing its dose. The inventors of the present invention have found that the bioavailability of dimethyl fumarate may be increased by modulating the conversion of dimethyl fumarate to monomethyl fumarate through an enzyme modulator. Alternatively, the bioavailability may be increased by the use of a permeation enhancer in the formulation. The present invention relates to a pharmaceutical composition of dimethyl fumarate comprising an enzyme modulator and/or a permeation enhancer, which provide increased bioavailability of dimethyl fumarate. The increased bioavailability may lead to dose reduction of dimethyl fumarate and hence reduced adverse effects. The inventors of the present invention have further found that the addition of an alcohol-resistant polymer in the coating composition provides a relatively high degree of alcohol resistance.

### SUMMARY OF THE INVENTION

The present invention relates to an oral pharmaceutical composition comprising dimethyl fumarate; an enzyme modulator or a permeation enhancer or both; and one or more pharmaceutically acceptable excipients. It further relates to a pulsatile release pharmaceutical composition comprising dimethyl fumarate and one or more pharmaceutically acceptable excipients. The compositions of the present invention can be administered at a lower dose as compared to the recommended daily dose of Tecfidera®. Further, the compositions of the present invention have a reduced food effect and reduced potential for alcohol-induced dose dumping.

### DETAILED DESCRIPTION OF THE INVENTION

A first aspect of the present invention provides an oral pharmaceutical composition comprising dimethyl fumarate; an enzyme modulator or a permeation enhancer or both; and one or more pharmaceutically acceptable excipients.

According to one embodiment of this aspect, the enzyme modulator is an esterase inhibitor or an esterase inducer.

According to another embodiment of this aspect, the esterase inhibitor is selected from the group comprising EDTA and its salts, such as sodium EDTA and disodium EDTA; sodium lauryl sulfate; Tween® 20; polyoxyl 40 stearate; polyoxyl 35 castor oil; ascorbic acid; lecithin; polyoxyl 40 hydrogenated castor oil; Triton® X-100; poloxamer 188; Tween® 80; PEG 200; PEG 400; PEG 6000; PEG 4000; sodium alginate; mannitol; lactose; and mixtures thereof.

According to another embodiment of this aspect, the esterase inducer is selected from calcium salts.

According to another embodiment of this aspect, the pharmaceutically acceptable excipients comprise one or more of fillers, binders, disintegrants, lubricants, and glidants.

According to another embodiment of this aspect, the composition further comprises a release modifier.

According to another embodiment of this aspect, the release modifier is a rate-controlling agent.

According to another embodiment of this aspect, the composition is coated with an enteric polymer.

According to another embodiment of this aspect the composition is coated with an alcohol-resistant polymer.

According to another embodiment of this aspect, the pharmaceutical composition is resistant to dose dumping in the presence of alcohol.

According to another embodiment of this aspect, the composition is present in the form of tablets, capsules, powder, caplets, beads, pellets, pellets in capsules, granules, granules in capsules, minitablets, minitablets in capsules, or sachet.

According to another embodiment of this aspect, the composition is administered one, two, or three times a day.

According to another embodiment of this aspect, dimethyl fumarate is present in an amount which is at least about 10% lower than the amount of dimethyl fumarate present in the formulation marketed under the trade name Tecfidera®.

According to another embodiment of this aspect, dimethyl fumarate is present in an amount which is at least about 15% lower than the amount of dimethyl fumarate present in the formulation marketed under the trade name Tecfidera®.

According to another embodiment of this aspect, dimethyl fumarate is present in an amount which is at least about 20% lower than the amount of dimethyl fumarate present in the formulation marketed under the trade name Tecfidera®.

According to another embodiment of this aspect, dimethyl fumarate is present in an amount which is at least about 25% lower than the amount of dimethyl fumarate present in the formulation marketed under the trade name Tecfidera®.

According to another embodiment of this aspect, dimethyl fumarate is present in an amount which is at least about 30% lower than the amount of dimethyl fumarate present in the formulation marketed under the trade name Tecfidera®.

According to another embodiment of this aspect, dimethyl fumarate is present in an amount which is at least about 35% lower than the amount of dimethyl fumarate present in the formulation marketed under the trade name Tecfidera®.

According to another embodiment of this aspect, dimethyl fumarate is present in an amount which is at least about 40% lower than the amount of dimethyl fumarate present in the formulation marketed under the trade name Tecfidera®.

According to another embodiment of this aspect, dimethyl fumarate is present in an amount that reduces the side effects associated with a high dose composition of dimethyl fumarate.

According to another embodiment of this aspect, dimethyl fumarate is present in an amount that reduces the side effects associated with the dimethyl fumarate formulation marketed under the trade name Tecfidera®.

According to another embodiment of this aspect, the pharmaceutical composition, when administered orally at a low dose, provides equivalent efficacy in comparison to the dimethyl fumarate formulation marketed under the trade name Tecfidera®.

According to another embodiment of this aspect, the low dose is about 432 mg per day, about 408 mg per day, about 384 mg per day, about 360 mg per day, about 336 mg per day, about 312 mg per day, or about 288 mg per day.

According to another embodiment of this aspect, the low dose is 432 mg per day, 408 mg per day, 384 mg per day, 360 mg per day, 336 mg per day, 312 mg per day, or 288 mg per day.

According to another embodiment of this aspect, the low dose is 432 mg per day administered as 216 mg capsules twice a day; the low dose is 408 mg per day administered as 204 mg capsules twice a day; the low dose is 384 mg per day administered as 192 mg capsules twice a day; the low dose is 360 mg per day administered as 180 mg capsules twice a day; the low dose is 336 mg per day administered as 168 mg capsules twice a day; the low dose is 312 mg per day administered as 156 mg capsules twice a day; or the low dose is 288 mg per day administered as 144 mg capsules twice a day.

According to another embodiment of this aspect, the pharmaceutical composition, when administered orally, provides one or both of rate and extent of absorption of dimethyl fumarate equal to or greater than that obtained by a dimethyl fumarate formulation marketed under the trade name Tecfidera®.

According to another embodiment of this aspect, the pharmaceutical composition, when administered orally, provides a reduced food effect.

A second aspect of the present invention provides an oral pulsatile-release pharmaceutical composition comprising dimethyl fumarate and one or more pharmaceutically acceptable excipients.

According to one embodiment of this aspect, the composition comprises one or more modified release components or a combination of an immediate release component and a modified release component.

According to another embodiment of this aspect, the modified release component is an extended release component, a delayed release component, a delayed extended release component, a colon-targeted release component, or a combination thereof.

According to another embodiment of this aspect, the composition comprises a delayed release component and a colon-targeted release component.

According to another embodiment of this aspect, the pharmaceutically acceptable excipients comprise one or more of fillers, binders, disintegrants, lubricants, and glidants.

According to another embodiment of this aspect, the composition further comprises an enzyme modulator or a permeation enhancer or both.

According to another embodiment of this aspect, the composition is coated with an enteric polymer.

According to another embodiment of this aspect, the composition is coated with an alcohol-resistant polymer.

According to another embodiment of this aspect, the composition is resistant to dose dumping in the presence of alcohol.

According to another embodiment of this aspect, the composition is present in the form of tablets, capsules, powder, caplets, beads, pellets, pellets in capsules, granules, granules in capsules, minitablets, minitablets in capsules, or sachet.

According to another embodiment of this aspect, the composition is administered one, two, or three times a day.

According to another embodiment of this aspect, dimethyl fumarate is present in an amount which is at least about 10% lower than the amount of dimethyl fumarate present in the formulation marketed under the trade name Tecfidera®.

According to another embodiment of this aspect, dimethyl fumarate is present in an amount which is at least about 15% lower than the amount of dimethyl fumarate present in the formulation marketed under the trade name Tecfidera®.

According to another embodiment of this aspect, dimethyl fumarate is present in an amount which is at least about 20% lower than the amount of dimethyl fumarate present in the formulation marketed under the trade name Tecfidera®.

According to another embodiment of this aspect, dimethyl fumarate is present in an amount which is at least about 25% lower than the amount of dimethyl fumarate present in the formulation marketed under the trade name Tecfidera®.

According to another embodiment of this aspect, dimethyl fumarate is present in an amount which is at least about 30% lower than the amount of dimethyl fumarate present in the formulation marketed under the trade name Tecfidera®.

According to another embodiment of this aspect, dimethyl fumarate is present in an amount which is at least about 35% lower than the amount of dimethyl fumarate present in the formulation marketed under the trade name Tecfidera®.

According to another embodiment of this aspect, dimethyl fumarate is present in an amount which is at least about 40% lower than the amount of dimethyl fumarate present in the formulation marketed under the trade name Tecfidera®.

According to another embodiment of this aspect, dimethyl fumarate is present in an amount that reduces the side effects associated with a high dose composition of dimethyl fumarate.

According to another embodiment of this aspect, the pharmaceutical composition, when administered orally at a low dose, provides equivalent efficacy in comparison to the dimethyl fumarate formulation marketed under the trade name Tecfidera®.

According to another embodiment of this aspect, the low dose is about 432 mg per day, about 408 mg per day, about 384 mg per day, about 360 mg per day, about 336 mg per day, about 312 mg per day, or about 288 mg per day.

According to another embodiment of this aspect, the low dose is 432 mg per day, 408 mg per day, 384 mg per day, 360 mg per day, 336 mg per day, 312 mg per day, or 288 mg per day.

According to another embodiment of this aspect, the low dose is 432 mg per day administered as 216 mg capsules twice a day; the low dose is 408 mg per day administered as 204 mg capsules twice a day; the low dose is 384 mg per day administered as 192 mg capsules twice a day; the low dose is 360 mg per day administered as 180 mg capsules twice a day; the low dose is 336 mg per day administered as 168 mg capsules twice a day; the low dose is 312 mg per day administered as 156 mg capsules twice a day; the low dose is 288 mg per day administered as 144 mg capsules twice a day.

According to another embodiment of this aspect, the pharmaceutical composition, when administered orally, provides one or both of the rate and extent of absorption of dimethyl fumarate equal to or greater than that obtained by a dimethyl fumarate formulation marketed under the trade name Tecfidera®.

According to another embodiment of this aspect, the pharmaceutical composition, when administered orally, provides a reduced food effect.

A third aspect of the present invention provides a method of treating a disease condition requiring dimethyl fumarate therapy, said method comprising orally administering to a subject in need thereof an oral pharmaceutical composition according to the present invention.

According to one embodiment of this aspect, the disease condition is multiple sclerosis or psoriasis.

According to another embodiment of this aspect, the disease condition is multiple sclerosis.

A fourth aspect of the present invention provides a method of treating multiple sclerosis, said method comprising orally administering to a subject in need thereof dimethyl fumarate at a low dose of about 288 mg per day to about 384 mg per day

According to one embodiment of this aspect, the dimethyl fumarate is administered at a low dose of about 288 mg per day to about 378 mg per day

According to another embodiment of this aspect, the dimethyl fumarate is administered at a low dose of about 360 mg per day to about 378 mg per day.

According to another embodiment of this aspect, the dimethyl fumarate is administered at a low dose of about 360 mg per day.

According to another embodiment of this aspect, dimethyl fumarate, when administered at low dose to healthy subjects under fasting conditions, provides a mean Cₘₐₓ/dose in the range of about 11.23 to about 16.85 ng/mL/mg.

According to another embodiment of this aspect, the dimethyl fumarate, when administered at low dose to healthy subjects under fasting conditions, provides a mean AUC/dose in the range of about 18.42 to about 27.63 ng·hr/mL/mg.

The term “low dose,” as used herein, refers to a therapeutically effective dose of dimethyl fumarate, which dose is less than the usual or the conventional dose required to produce the therapeutic effect. The low dose is at least 10%, at least 15%, at least 20%, at least 25%, at least 30%, at least 35%, or at least 40% lower than the currently approved starting dose of 240 mg per day and maintenance dose of 480 mg per day. Preferably, the low dose is about 15% or about 20% or about 25% lower than the currently approved dose of 480 mg per day. The low dose is about 432 mg per day, about 408 mg per day, about 384 mg per day, about 360 mg per day, about 336 mg per day, about 312 mg per day, or about 288 mg per day. The low dose is about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, or about 280 mg per day. Preferably the low dose is 432 mg, 430 mg, 428 mg, 426 mg, 424 mg, 422 mg, 420 mg, 418 mg, 416 mg, 414 mg, 412 mg, 410 mg, 408 mg, 406 mg, 404 mg, 402 mg, 400 mg, 398 mg, 396 mg, 394 mg, 392 mg, 390 mg, 388 mg, 386 mg, 384 mg, 382 mg, 380 mg, 378 mg, 376 mg, 374 mg, 372 mg, 370 mg, 368 mg, 366 mg, 364 mg, 362 mg, 360 mg, 358 mg, 356 mg, 354 mg, 352 mg, 350 mg, 348 mg, 346 mg, 344 mg, 342 mg, 340 mg, 338 mg, 336 mg, 334 mg, 332 mg, 330 mg, 328 mg, 326 mg, 324 mg, 322 mg, 320 mg, 318 mg, 316 mg, 314 mg, 312 mg, 310 mg, 308 mg, 306 mg, 304 mg, 302 mg, 300 mg, 298 mg, 296 mg, 294 mg, 292 mg, 290 mg, or 288 mg per day. The low dose of dimethyl fumarate is administered by giving the composition of the present invention twice daily. The low dose of 432 mg per day is administered as 216 mg capsules twice a day; the low dose of 408 mg per day is administered as 204 mg capsules twice a day; the low dose of 384 mg per day is administered as 192 mg capsules twice a day; the low dose of 360 mg per day is administered as 180 mg capsules twice a day; the low dose of 336 mg per day is administered as 168 mg capsules twice a day; the low dose of 312 mg per day is administered as 156 mg capsules twice a day; or the low dose of 288 mg per day is administered as 144 mg capsules twice a day.

The composition of the present invention contains dimethyl fumarate in an amount lower than that present in the formulation marketed under the trade name Tecfidera®. The dimethyl fumarate is present in the composition in an amount which is at least about 10%, at least about 15%, at least about 20%, at least about 25%, at least about 30%, at least about 35%, or at least about 40% lower than the amount of dimethyl fumarate present in Tecfidera®. Tecfidera® is available in two strengths which contain 120 mg and 240 mg of dimethyl fumarate. Hence, dimethyl fumarate is present in the composition in an amount of about 72 mg to about 108 mg for lower strength or in an amount of about 144 mg to about 216 mg for higher strength. The dimethyl fumarate is present in the composition in an amount of about 216 mg, about 204 mg, about 192 mg, about 180 mg, about 168 mg, about 156 mg, or about 144 mg. For the lower strength, the dimethyl fumarate is present in the composition in an amount of about 108 mg, about 102 mg, about 96 mg, about 90 mg, about 84 mg, about 78 mg, or about 72 mg.

The term “enzyme modulator,” as used herein, refers to agents which modulate the activity of the esterase enzyme. They include esterase inhibitors and esterase inducers.

The term “permeation enhancer,” as used herein, refers to agents that improve the rate of transport of a pharmacologically active agent across the mucosal surface. Typically, a permeation enhancer increases the permeability of mucosal tissue to a therapeutic agent.

The term “pharmaceutically acceptable excipients,” as used herein, includes any physiologically inert additives that are routinely used in pharmaceutical compositions. These may be selected from the group comprising diluents, binders, disintegrants, lubricants/glidants/anti-adherents, and mixtures thereof.

After oral administration, dimethyl fumarate is extensively metabolized by esterases and is converted to its active metabolite, monomethyl fumarate. The inventors of the present invention have found that the bioavailability of dimethyl fumarate compositions may be increased by modulating the activity of the esterase enzymes.

The first approach to increasing the bioavailability of dimethyl fumarate is the use of an enzyme inhibitor in the composition. As per the literature, dimethyl fumarate is unstable in (porcine) intestinal fluid due to the presence of esterase, suggesting the potential of dimethyl fumarate to be degraded also in a human GI tract before absorption. Literature suggests a passive absorption mechanism as a means of transport for fumarate. The permeability coefficient for dimethyl fumarate is significantly higher than that of monomethyl fumarate (half-life of 0.5 to 1.4 hours). Thus, by inhibiting the conversion of dimethyl fumarate to monomethyl fumarate in the intestine, absorption of dimethyl fumarate may be increased. In such a case, the maximum conversion will occur in the blood, and thus higher monomethyl fumarate plasma concentration can be achieved. Examples of esterase inhibitors include EDTA and salts such as sodium EDTA and disodium EDTA; sodium lauryl sulfate; Tween® 20; polyoxyl 40 stearate; polyoxyl 35 castor oil; ascorbic acid; lecithin; polyoxyl 40 hydrogenated castor oil; Triton® X-100; poloxamer 188; Tween® 80; PEG 200; PEG 400; PEG 6000; PEG 4000; sodium alginate; mannitol; lactose; and mixtures thereof.

Another approach to increasing the bioavailability of dimethyl fumarate is the use of an enzyme inducer in the composition. Examples of esterase inducers include calcium salts such as tricalcium phosphate.

Another approach to increasing the bioavailability of dimethyl fumarate is the use of a permeation enhancer in the composition. The permeation enhancer increases the rate at which the therapeutic agent permeates through membranes and enters the bloodstream, thus higher monomethyl fumarate plasma concentration can be achieved. Examples of permeation enhancers include synthetic surfactants which include cationic surfactants such as cetyl trimethylammonium bromide, cetyl pyridinium chloride, benzalkonium chloride, anionic surfactants such as sodium lauryl sulfate, sodium dodecyl sulfate, sodium laurate, nonionic surfactants such as laureth 9, dodecylmaltoside, Tween®, Span®, Brij®, Myrj®, polaxamer, sucrose esters; bile salts and other steroidal detergents such as sodium cholate, sodium deoxycholate, sodium glycocholate, sodium glycodeoxycholate, sodium taurocholate, sodium taurodeoxycholate, sodium tauroglycocholate, sodium glycodihydrofusidate, sodium taurodihydrofusidate, sodium ursocholate, saponins; fatty acid and derivatives such as lauric acid, oleic acid, caprylic acid, neodecanoic acid, elaidic acid; phospholipids such as phosphatidylcholine, lysophosphatidylcholine, didecanoylphosphatidylcholine; chelators such as sodium EDTA, citric acid, sodium salicylate, methoxy salicylates, polyacrylates; cyclodextrins such as α-, β-, and γ-cyclodextrin, hydroxypropyl-β-cyclodextrin, methylated-β-cyclodextrin; positively charged polymers such as chitosan, trimethylchitosan, chitosan-4-thioglycolic acid, chitosan-4-thio-butylamidine, dextran sulfate, poly-L-arginine, L-lysine, and mixtures thereof.

Another approach to increasing the bioavailability of dimethyl fumarate is to formulate a dimethyl fumarate formulation as a pulsatile release composition.

The pharmaceutical composition of the present invention may comprise an immediate release component, a modified release component, or a combination thereof. The modified release component is an extended release component, delayed release component, delayed extended release component, colon-targeted release component, or a combination thereof. In an embodiment, the composition comprises a delayed release component and a colon-targeted release component.

In order to provide a relatively high degree of alcohol resistance, the pharmaceutical composition of the present invention comprises an alcohol-resistant polymer. The alcohol-resistant polymer may be present in the core or in the coating. Preferably, the alcohol-resistant polymer is present in the enteric coating, in the overcoat over the enteric coating, as a separate coating layer over the drug core, over the seal coat, or over the enteric coat. Suitable alcohol-resistant polymers are selected from the group comprising natural gums such as sodium alginate, guar gum, locust bean gum, acacia, pectin, psyllium, and karaya; synthetic polymer gums such as carboxymethyl cellulose, polyethylene glycol, polyvinylpyrrolidone, polyvinyl alcohol, and derivatives; cellulose acetate phthalate; hypromellose phthalate; Eudragit® S; a mixture of Eudragit® L 30 D-55 and Eudragit® L 100-55; and mixtures thereof.

The pharmaceutically acceptable excipients may be one or more of diluents, binders, disintegrants, lubricants, and glidants.

Suitable diluents are selected from the group comprising lactose, e.g., lactose anhydrous or lactose monohydrate; cellulose, e.g., microcrystalline cellulose, co-processed microcrystalline cellulose, or powdered cellulose; starch, e.g., pregelatinized starch, maize starch, rice starch, potato starch, or wheat starch; sugar alcohols, e.g., mannitol, sorbitol, xylitol, or erythritol; inorganic salts, e.g., calcium carbonate, calcium phosphate, calcium sulfate, dibasic calcium phosphate, dibasic calcium phosphate anhydrate, dibasic calcium phosphate dihydrate, or tribasic calcium phosphate; and mixtures thereof. The diluents may also act as binders.

Suitable binders are selected from the group comprising povidone, copovidone, hydroxypropylmethyl cellulose, hydroxypropyl cellulose, hydroxyethyl cellulose, methyl cellulose, ethyl cellulose, carboxymethyl cellulose sodium, xanthan gum, gum acacia, gum arabic, tragacanth, sorbitol, dextrose, sucrose, lactose, mannitol, gelatin, pullulan, sodium alginate, propylene glycol, polyvinyl alcohol, corn starch, modified corn starch, maize starch, pregelatinized starch, methacrylates, carboxyvinyl polymers, waxes, and mixtures thereof.

Suitable disintegrants are selected from the group comprising croscarmellose sodium, hydroxypropyl cellulose, crospovidone, low substituted hydroxypropyl cellulose, microcrystalline cellulose, carboxymethyl cellulose sodium, carboxymethyl cellulose calcium, gums, alginic acid or alginates, starch, corn starch, pregelatinized starch, modified starch, sodium starch glycolate, carboxymethyl starch, polyacrylates, and mixtures thereof.

Suitable lubricants are selected from the group comprising stearic acid, magnesium stearate, calcium stearate, zinc stearate, sodium stearyl fumarate, polyethylene glycol, talc, hydrogenated vegetable oils, fatty acids, waxes, and mixtures thereof.

Suitable glidants or anti-sticking agents are selected from the group comprising talc, silicon dioxide, colloidal silicon dioxide (colloidal anhydrous silica), calcium silicate, magnesium silicate, hydrated silica, and mixtures thereof.

The pharmaceutical composition of the present invention may further comprise a release modifier such as a rate-controlling agent. Suitable rate-controlling agents may be selected from the group comprising hydrophilic polymers, hydrophobic polymers, water swellable polymers, other hydrophobic materials, and mixtures thereof. Examples of rate-controlling agents include, but are not limited to, hydroxypropylmethyl cellulose, microcrystalline cellulose, hydroxypropyl cellulose, methyl cellulose, sodium carboxymethyl cellulose, hydroxyethyl cellulose, carboxymethyl ethyl cellulose, ethyl cellulose, cellulose acetate, cellulose nitrate, other cellulose derivatives, methacrylic acid copolymer, e.g., polymethacrylic copolymer, poloxamers, polyoxyethylene stearate, polyvinylpyrrolidone, polyvinylpyrrolidone-polyvinylacetate copolymer, polyvinyl alcohol, polyethylene oxide, gums (e.g., xanthan gum, tragacanth gum, gum karaya, guar gum, acacia gum, and locust bean gum), fatty acids, fatty acid esters, alkyl alcohols, wax, shellac, and mixtures thereof.

The pharmaceutical composition of the present invention can be prepared by processes known in the art like wet granulation, dry granulation, or direct compression.

The pharmaceutical composition of the present invention can be present in the form of tablets, capsules, powder, caplets, beads, pellets, pellets in capsules, granules, granules in capsules, minitablets, minitablets in capsules, sachet, or other dosage forms suitable for oral administration. Alternatively, the pharmaceutical composition can be administered as a sprinkle dosage form or via nasogastric tube. The dimethyl fumarate compositions of the present invention have a lower amount of drug and thus have a smaller size as compared to the currently marketed formulations, thus leading to improved patient compliance.

The pharmaceutical composition of the present invention may be coated or uncoated, including but not limited to, seal coating, extended release coating, enteric (delayed release) coating, colon-targeting coating, alcohol-resistant coating, or a combination thereof. Additional excipients such as film-forming polymers, plasticizers, anti-adherents or anti-tacking agents, antifoaming agents, opacifiers, colorants, pigments, and polishing agents may be used in coatings. Further, an enzyme modulator may be present in the coating.

Suitable extended release coating polymers are selected from the group comprising hydroxypropylmethyl cellulose, microcrystalline cellulose, hydroxypropyl cellulose, methyl cellulose, sodium carboxymethyl cellulose, hydroxyethyl cellulose, carboxymethyl ethyl cellulose, ethyl cellulose, cellulose acetate, cellulose nitrate, other cellulose derivatives, methacrylic acid copolymer, polymethacrylic copolymer, poloxamers, polyoxyethylene stearate, polyvinylpyrrolidone, polyvinylpyrrolidone-polyvinylacetate copolymer, polyvinyl alcohol, polyethylene oxide, gums, fatty acids, fatty acid esters, alkyl alcohols, wax, shellac, and mixtures thereof.

Suitable enteric coating polymers are selected from the group comprising cellulosic polymers like cellulose acetate phthalates, cellulose acetate trimellitates, hydroxypropyl methylcellulose phthalates, polyvinyl acetate phthalates, methacrylic acid polymers and copolymers such as methacrylic acid and ethyl acrylate copolymer dispersion (Eudragit® L 30 D-55), methyl acrylate-methyl methacrylate-methacrylic acid copolymer (Eudragit® FS 30 D), a methacrylic acid-methyl methacrylate copolymer, a methacrylic acid-ethyl acetate copolymer, and mixtures thereof.

Suitable colon-targeting coating polymers are selected from the group comprising guar gum, cyclodextrin amylase, locust bean gum, shellac, ethyl cellulose, cellulose acetate phthalate, hydroxypropylmethyl cellulose, Eudragit®, polyvinylacetate phthalate, and mixtures thereof.

Suitable film-forming polymers are selected from the group comprising hydroxypropylmethyl cellulose, ethyl cellulose, methyl cellulose, hydroxyethyl cellulose, hydroxypropyl cellulose, sodium carboxymethyl cellulose, cellulose acetate, hydroxypropylmethyl cellulose phthalate, cellulose acetate trimellitate, methacrylic acid copolymers, e.g., Eudragit®, polyvinylpyrrolidone, polyvinylalcohol, polyethylene glycol, and mixtures thereof. Other suitable film-forming polymers which are known in the art may also be used. Many suitable film coating products which are commercially available, e.g., Opadry® and Opaglos®, may be used.

Suitable plasticizers include propylene glycol, triethyl citrate, tributyl citrate, dibutyl sebacate, acetyl tributyl citrate, glyceryl monostearate, triacetin, polyethylene glycol, diethyl phthalate, acetylated monoglycerides, diacetylated monoglycerides, cetyl alcohol, and mixtures thereof.

Suitable anti-adherents or anti-tacking agents are selected from the group comprising talc, kaolin, finely divided silicon dioxide, glyceryl monostearate, aluminum hydrate, magnesium stearate, and mixtures thereof.

Suitable antifoaming agents are selected from the group comprising simethicone, polydimethylsiloxane, other silicones, stearates, alcohols, glycols, and mixtures thereof.

Suitable opacifiers are selected from the group comprising titanium dioxide, manganese dioxide, iron oxide, silicon dioxide, and mixtures thereof.

Suitable coloring agents are selected from FDA-approved colorants such as iron oxide, lake of tartrazine, allura red, titanium dioxide, and mixtures thereof.

Suitable polishing agents are selected from the group comprising polyethylene glycols of various molecular weights or mixtures thereof, talc, surfactants (e.g. glycerol monostearate and poloxamers), fatty alcohols (e.g. stearyl alcohol, cetyl alcohol, lauryl alcohol, and myristyl alcohol), waxes (e.g. carnauba wax, candelilla wax and, white wax), and mixtures thereof.

Various solvents that may be employed during the preparation of the pharmaceutical composition of the present invention are selected from the group comprising water, methyl alcohol, ethyl alcohol, isopropyl alcohol, n-butyl alcohol, acetone, acetonitrile, chloroform, methylene chloride, and mixtures thereof.

The coating may be carried out by using any conventional coating techniques known in the art, such as spray coating in a conventional coating pan or fluidized bed processor, or dip coating.

The pharmaceutical composition according to the present invention may be administered at least once a day. Preferably, the pharmaceutical composition is administered twice a day in a dose which is less than the conventionally administered daily dose.

The pharmaceutical composition according to the present invention may be used for the treatment of a disease condition requiring dimethyl fumarate therapy. The disease condition requiring dimethyl fumarate therapy may be selected from the group consisting of diseases and conditions involving immunological, autoimmune, and/or inflammatory processes including psoriasis; asthma and chronic obstructive pulmonary diseases; cardiac insufficiency including left ventricular insufficiency, myocardial infarction, and angina pectoris; mitochondrial and neurodegenerative diseases such as Parkinson's disease, Alzheimer's disease, Huntington's disease, retinopathia pigmentosa, and mitochondrial encephalomyopathy; transplantation; autoimmune diseases including multiple sclerosis (MS); ischemia and reperfusion injury; inflammatory bowel diseases such as Crohn's disease and ulcerative colitis; arthritis; juvenile-onset diabetes; Hashimoto's thyroiditis; Grave's disease; systemic Lupus erythematodes (SLE); Sjogren's syndrome; pernicious anemia; chronic active (lupoid) hepatitis; and others. Preferably, the pharmaceutical composition according to the present invention is used for the treatment of multiple sclerosis or psoriasis. More preferably, the pharmaceutical composition according to the present invention is used for the treatment of multiple sclerosis.

The pharmaceutical composition of the present invention when administered orally at a low dose provides equivalent efficacy in comparison to the dimethyl fumarate formulation marketed under the trade name Tecfidera®.

The pharmaceutical composition of the present invention, when administered orally at a low dose to a subject under fasting conditions, provides the ratio of mean Cₘₐₓ to dose in the range of about 11.23 to about 16.85 ng/mL/mg and the ratio of mean AUC to dose in the range of about 18.42 to about 27.63 hr*ng/mL/mg.

The pharmaceutical composition of the present invention is resistant to dose dumping in presence of alcohol, such that, when tested in 900 mL of hydro-alcoholic media (0.1N HCl with 5% to 40% ethanol) in USP dissolution apparatus II, it releases not more than 25% of the total drug content after 2 hours.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Examples 1 and 2: Composition Comprising an Enzyme Inducer

| | Example 1 | Example 2 |
|---|---|---|
| Ingredients | Percent (%) w/w | Percent (%) w/w |
| Core tablet | | |
| Dimethyl fumarate | 57.59 | 73.07 |
| Microcrystalline cellulose | 10.53 | 2.13 |
| Tricalcium phosphate | 4.08 | 4.57 |
| Hydroxypropylmethyl cellulose | 8.16 | 4.57 |
| Colloidal silicon dioxide | 0.41 | — |
| Magnesium stearate | 0.82 | 0.91 |
| Seal coat | | |
| Methacrylic acid copolymer—type A | 1.88 | 1.31 |
| Triethyl citrate | 0.56 | 0.39 |
| Isopropyl alcohol | q.s. | q.s. |
| Enteric coat | | |
| Methacrylic acid copolymer | 9.38 | 7.66 |
| dispersion | | |
| Triethyl citrate | 1.88 | 1.53 |
| Talc | 4.68 | 3.84 |
| Simethicone | 0.03 | 0.02 |
| Purified water | q.s. | q.s. |

### Manufacturing Procedure:

### Stage I: Tablet Compression

1. Dimethyl fumarate, microcrystalline cellulose, colloidal silicon dioxide, tricalcium phosphate, and hydroxypropylmethyl cellulose were blended in a blender.
2. Magnesium stearate was added to the step 1 material and blended.
3. The blend of step 2 was compressed into mini tablets.

### Stage II: Seal Coating

4. Methacrylic acid copolymer—type A was dissolved in isopropyl alcohol under mechanical stirring until a clear dispersion was formed, followed by the addition of triethyl citrate.
5. The core mini tablets of step 3 were coated with the dispersion of step 4.

### Stage III: Enteric Coating

6. Triethyl citrate and talc were dispersed in purified water using a homogenizer to obtain a dispersion.
7. A simethicone emulsion was added into the dispersion of step 6.
8. The dispersion of step 7 was added into a methacrylic acid copolymer dispersion under continuous stirring, and then filtered.
9. The seal-coated mini tablets of step 5 were coated with the dispersion of step 8 to achieve the desired weight buildup.

### Stage IV: Capsule Filling

10. The enteric-coated mini tablets of step 9 were filled into hard gelatin capsules.

### Examples 3, 4, and 5: Composition Comprising an Enzyme Inhibitor

| | Example 3 | Example 4 | Example 5 |
|---|---|---|---|
| | Percent | Percent | Percent |
| Ingredients | (%) w/w | (%) w/w | (%) w/w |
| Core tablet | | | |
| Dimethyl fumarate | 57.59 | 31.58 | 73.07 |
| Microcrystalline cellulose | 10.53 | 32.46 | 2.74 |
| Disodium EDTA | 4.08 | 4.08 | 4.26 |
| Hydroxypropylmethyl cellulose | 8.16 | 12.24 | 4.26 |
| Colloidal silicon dioxide | 0.41 | 0.82 | — |
| Magnesium stearate | 0.82 | 0.41 | 0.91 |
| Seal coat | | | |
| Methacrylic acid copolymer—type A | 1.88 | 1.88 | 1.31 |
| Triethyl citrate | 0.56 | 0.56 | 0.39 |
| Isopropyl alcohol | q.s. | q.s. | q.s. |
| Enteric coat | | | |
| Methacrylic acid copolymer | 9.38 | 9.38 | 7.66 |
| dispersion | | | |
| Triethyl citrate | 1.88 | 1.88 | 1.53 |
| Talc | 4.68 | 4.68 | 3.85 |
| Simethicone | 0.03 | 0.03 | 0.02 |
| Purified water | q.s. | q.s. | q.s. |

### Manufacturing Procedure:

The capsules of Examples 3, 4, and 5 were prepared by following the same procedure as for Example 1, with the exception that an enzyme inhibitor (disodium EDTA) was added in place of an enzyme inducer (tricalcium phosphate).

### Example 6: Composition Comprising an Alcohol-Resistant Polymer

| Ingredients | Percent (%) w/w |
|---|---|
| Core tablet | |
| Dimethyl fumarate | 58.00 |
| Microcrystalline cellulose | 2.22 |
| Croscarmellose sodium | 4.83 |
| Disodium EDTA | 4.83 |
| Sodium lauryl sulphate | 6.24 |
| Colloidal silicon dioxide | 0.79 |
| Magnesium stearate | 0.42 |
| Seal coat | |
| Methacrylic acid copolymer—type A | 1.79 |
| Triethyl citrate | 0.53 |
| Isopropyl alcohol | q.s. |
| Enteric coat | |
| Methacrylic acid copolymer dispersion | 11.69 |
| Triethyl citrate | 2.34 |
| Talc | 5.85 |
| Simethicone | 0.03 |
| Guar gum | 0.44 |
| Purified water | q.s. |

### Manufacturing Procedure:

### Stage I: Tablet Compression

1. Dimethyl fumarate, microcrystalline cellulose, croscarmellose sodium, disodium EDTA, sodium lauryl sulphate, and colloidal silicon dioxide were blended in a blender.
2. Magnesium stearate was added to the step 1 material and blended.
3. The blend of step 2 was compressed into mini tablets.

### Stage II: Seal Coating

4. Methacrylic acid copolymer—type A was dissolved in isopropyl alcohol under mechanical stirring until a clear dispersion was formed, followed by the addition of triethyl citrate.
5. The core mini tablets of step 3 were coated with the dispersion of step 4.

### Stage III: Enteric Coating

6. Triethyl citrate and talc were dispersed in purified water using a homogenizer to obtain a dispersion.
7. A simethicone emulsion was added into the dispersion of step 6, followed by the addition of guar gum.
8. The dispersion of step 7 was added into a methacrylic acid copolymer dispersion under continuous stirring, and then filtered.
9. The seal-coated mini tablets of step 5 were coated with the dispersion of step 8 to achieve the desired weight buildup.

### Stage IV: Capsule Filling

10. The enteric-coated mini tablets of step 9 were filled into hard gelatin capsules.

### Example 7: Composition Comprising an Alcohol-Resistant Polymer

| Ingredients | Percent (%) w/w |
|---|---|
| Core tablet | |
| Dimethyl fumarate | 58.00 |
| Microcrystalline cellulose | 2.22 |
| Croscarmellose sodium | 4.83 |
| Disodium EDTA | 4.83 |
| Sodium lauryl sulphate | 6.24 |
| Colloidal silicon dioxide | 0.79 |
| Magnesium stearate | 0.42 |
| Seal coat | |
| Methacrylic acid copolymer—type A | 1.79 |
| Triethyl citrate | 0.53 |
| Isopropyl alcohol | q.s. |
| Enteric coat | |
| Methacrylic acid copolymer dispersion | 11.69 |
| Triethyl citrate | 2.34 |
| Talc | 5.85 |
| Simethicone | 0.03 |
| Purified water | q.s. |
| Overcoat | |
| Guar gum | 0.44 |
| Purified water | q.s. |

### Manufacturing Procedure:

### Stage I: Tablet Compression

1. Dimethyl fumarate, microcrystalline cellulose, croscarmellose sodium, disodium EDTA, sodium lauryl sulphate, and colloidal silicon dioxide were blended in a blender.
2. Magnesium stearate was added to the step 1 material and blended.
3. The blend of step 2 was compressed into mini tablets.

### Stage II: Seal Coating

4. Methacrylic acid copolymer—type A is dissolved in isopropyl alcohol under mechanical stirring until a clear dispersion is formed, followed by the addition of triethyl citrate.
5. The core mini tablets of step 3 are coated with the dispersion of step 4.

### Stage III: Enteric Coating

6. Triethyl citrate and talc are dispersed in purified water using a homogenizer to obtain a dispersion.
7. A simethicone emulsion is added into the dispersion of step 6.
8. The dispersion of step 7 is added into a methacrylic acid copolymer dispersion under continuous stirring, and then filtered.
9. The seal-coated mini tablets of step 5 are coated with the dispersion of step 8 to achieve the desired weight buildup.

### Stage IV: Over Coating

10. A coating dispersion is prepared by dispersing guar gum in purified water.
11. The enteric-coated mini tablets of step 9 are coated with the dispersion of step 10 to achieve the desired weight buildup.

### Stage V: Capsule Filling

12. The coated mini tablets of step 11 are filled into hard gelatin capsules.

### Example 8: Pulsatile Release Composition

| Ingredients | Percent (%) w/w |
|---|---|
| Core tablet | |
| Dimethyl fumarate | 64.61 |
| Microcrystalline cellulose | 2.47 |
| Croscarmellose sodium | 5.38 |
| Disodium EDTA | 5.38 |
| Sodium lauryl sulphate | 6.96 |
| Colloidal silicon dioxide | 0.87 |
| Magnesium stearate | 0.46 |
| Seal coat | |
| Methacrylic acid copolymer-type A | 1.72 |
| Methacrylic acid copolymer-type B | 1.72 |
| Triethyl citrate | 0.86 |
| Isopropyl alcohol | q.s. |
| Enteric coat | |
| Methacrylic acid copolymer dispersion | 5.31 |
| Triethyl citrate | 1.06 |
| Talc | 2.68 |
| Simethicone | 0.02 |
| Purified water | q.s. |
| Overcoat | |
| Guar gum | 0.50 |
| Purified water | q.s. |

### Manufacturing Procedure:

The capsules of Example 8 are prepared by following the same procedure as for Example 7.

### Example 9: Pulsatile Release Composition

| Ingredients | Percent (%) w/w |
|---|---|
| Core tablet | |
| Dimethyl fumarate | 65.84 |
| Microcrystalline cellulose | 2.52 |
| Croscarmellose sodium | 5.49 |
| Disodium EDTA (optional) | 5.49 |
| Sodium lauryl sulphate (optional) | 7.09 |
| Colloidal silicon dioxide | 0.89 |
| Magnesium stearate | 0.47 |
| Extended release coat (pulsatile) | |
| Ethyl cellulose | 1.10 |
| Methacrylic acid copolymer-type A | 6.86 |
| Triethyl citrate | 1.78 |
| Calcium stearate/magnesium | 1.10 |
| stearate | |
| Talc | 0.96 |
| Titanium dioxide | 0.41 |
| Dehydrated alcohol | q.s. |

### Manufacturing Procedure:

### Stage I: Tablet Compression

1. Dimethyl fumarate, microcrystalline cellulose, croscarmellose sodium, disodium EDTA, sodium lauryl sulphate, and colloidal silicon dioxide are blended in a blender.
2. Magnesium stearate was added to the step 1 material and blended.
3. The blend of step 2 was compressed into mini tablets.

### Stage II: Extended Release Coating

4. Ethyl cellulose and methacrylic acid copolymer (1:1) type A are dissolved in dehydrated alcohol until a clear dispersion is formed.
5. Triethyl citrate, calcium stearate/magnesium stearate, talc, and titanium dioxide are added into the step 4 dispersion.
6. The core mini tablets of step 3 are coated with the dispersion of step 5 to achieve the desired weight build-up.

### Stage III: Capsule Filling

7. The coated mini tablets of step 6 are filled into hard gelatin capsules.

### Example 10: Pulsatile Release Composition

| Ingredients | Percent (%) w/w |
|---|---|
| Core tablet | |
| Dimethyl fumarate | 58.45 |
| Microcrystalline cellulose | 2.24 |
| Croscarmellose sodium | 4.87 |
| Disodium EDTA (optional) | 4.04 |
| Sodium lauryl sulphate (optional) | 6.29 |
| Colloidal silicon dioxide | 0.79 |
| Magnesium stearate | 0.42 |
| Extended release coat (pulsatile) | |
| Ethyl cellulose | 9.74 |
| Hydroxypropylmethyl cellulose | 1.95 |
| Triethyl citrate | 0.97 |
| Isopropyl alcohol | q.s. |
| Purified water | q.s. |
| Enteric coat | |
| Methacrylic acid copolymer dispersion | 6.60 |
| Triethyl citrate | 1.32 |
| Talc | 2.30 |
| Simethicone | 0.02 |
| Purified water | q.s. |

### Manufacturing Procedure:

### Stage I: Tablet Compression

1. Dimethyl fumarate, microcrystalline cellulose, croscarmellose sodium, disodium EDTA, sodium lauryl sulphate, and colloidal silicon dioxide are blended in a blender.
2. Magnesium stearate is added to the step 1 material and blended.
3. The blend of step 2 is compressed into mini tablets.

### Stage II: Extended Release Coating

4. Ethyl cellulose is dissolved in isopropyl alcohol under mechanical stirring until a clear dispersion is formed.
5. Hydroxypropylmethyl cellulose is dissolved in water under mechanical stirring until a clear dispersion is formed.
6. The clear dispersion of step 4 is added to the clear dispersion of step 5 under continuous stirring, followed by the addition of triethyl citrate.
7. The core mini tablets of step 3 are coated with the dispersion of step 6 to achieve the desired weight build-up.

### Stage III: Enteric Coating

8. Triethyl citrate and talc are dispersed in purified water using a homogenizer to obtain a dispersion.
9. A simethicone emulsion is added into the dispersion of step 8.
10. The dispersion of step 9 is added into a methacrylic acid copolymer dispersion under continuous stirring, and then filtered.
11. The coated mini tablets of step 7 are coated with the dispersion of step 10 to achieve the desired weight buildup.

### Stage IV: Capsule Filling

12. The coated mini tablets of step 11 are filled into hard gelatin capsules.

### Example 11: Pulsatile Release Composition: Combination of Two Delayed Release Components (A and B)

| | | |
|---|---|---|
| | Component A | Component B |
| | Percent (%) | Percent (%) |
| Ingredients | w/w | w/w |

Core tablet

| | | |
|---|---|---|
| Dimethyl fumarate | 63.54 | 64.61 |
| Microcrystalline cellulose | 2.43 | 2.47 |
| Croscarmellose sodium | 5.30 | 5.38 |
| Disodium EDTA | 5.30 | 5.38 |
| Sodium lauryl sulphate | 6.84 | 6.96 |
| Colloidal silicon dioxide | 0.86 | 0.87 |
| Magnesium stearate | 0.46 | 0.46 |

Seal coat

| | | |
|---|---|---|
| Methacrylic acid copolymer-type A | 1.96 | 1.72 |
| Methacrylic acid copolymer-type B | — | 1.72 |
| Triethyl citrate | 0.59 | 0.86 |
| Isopropyl alcohol | q.s. | q.s. |

Enteric coat

| | | |
|---|---|---|
| Methacrylic acid copolymer dispersion | 7.18 | 5.31 |
| Triethyl citrate | 1.44 | 1.06 |
| Talc | 3.58 | 2.68 |
| Simethicone | 0.02 | 0.02 |
| Purified water | q.s. | q.s. |

Overcoat

| | | |
|---|---|---|
| Guar gum | 0.50 | 0.50 |
| Purified water | q.s. | q.s. |

### Manufacturing Procedure:

The capsule of Example 11 is prepared by combining the minitablets of components A and B, which are prepared by following the same procedure as for Examples 7 and 8, respectively, into hard gelatin capsules.

### Example 12: Colon-Targeting Composition

| Ingredients | Percent (%) w/w |
|---|---|
| Core tablet | |
| Dimethyl fumarate | 61.93 |
| Microcrystalline cellulose | 2.37 |
| Croscarmellose sodium | 5.16 |
| Disodium EDTA | 5.16 |
| Sodium lauryl sulphate | 6.67 |
| Colloidal silicon dioxide | 0.84 |
| Magnesium stearate | 0.44 |
| Colon-targeting coat | |
| Methacrylic acid copolymer-type B | 5.50 |
| Triethyl citrate | 2.75 |
| Isopropyl alcohol | q.s. |
| Enteric coat | |
| Methacrylic acid copolymer dispersion | 5.09 |
| Triethyl citrate | 1.02 |
| Talc | 2.55 |
| Simethicone | 0.02 |
| Purified water | q.s. |
| Overcoat | |
| Guar gum | 0.50 |
| Purified water | q.s. |

### Manufacturing Procedure:

The capsule of Example 12 is prepared by following the same procedure as for Example 7.

### Example 13: Pulsatile Release Composition

| Ingredients | Percent (%) w/w |
|---|---|
| Core tablet | |
| Dimethyl fumarate | 60.90 |
| Microcrystalline cellulose | 13.96 |
| Croscarmellose sodium | 5.08 |
| Colloidal silicon dioxide | 0.63 |
| Magnesium stearate | 0.63 |
| Seal coat | |
| Methacrylic acid copolymer-type A | 1.88 |
| Triethyl citrate | 0.56 |
| Isopropyl alcohol | q.s. |
| Enteric coat | |
| Methacrylic acid copolymer dispersion | 9.33 |
| Triethyl citrate | 1.87 |
| Talc | 4.66 |
| Simethicone | 0.03 |
| Purified water | q.s. |
| Overcoat | |
| Guar gum | 0.47 |
| Purified water | q.s. |

### Manufacturing Procedure:

The capsule of Example 13 is prepared by following the same procedure as for Example 7.

### Example 14: Composition Comprising an Enzyme Inhibitor

| Ingredients | Percent (%) w/w |
|---|---|
| Core tablets | |
| Dimethyl fumarate (192 mg | 65.60 |
| or 204 mg) | |
| Microcrystalline cellulose | 6.61 |
| Croscarmellose sodium | 5.47 |
| Sodium lauryl sulfate | 7.06 |
| Disodium EDTA | 1.37 |
| Colloidal silicon dioxide | 0.89 |
| Magnesium stearate | 0.47 |
| Seal coat | |
| Hydroxypropylmethyl cellulose | 2.62 |
| Isopropyl alcohol | q.s. |
| Purified water | q.s. |
| Enteric coat | |
| Methacrylic acid copolymer | 5.82 |
| dispersion | |
| Triethyl citrate | 1.17 |
| Talc | 2.91 |
| Simethicone | 0.02 |
| Purified water | q.s. |

### Manufacturing Procedure:

### Stage I: Tablet Compression

1. Dimethyl fumarate, microcrystalline cellulose, croscarmellose sodium, sodium lauryl sulfate, disodium EDTA, and colloidal silicon dioxide were blended in a blender.
2. Magnesium stearate was added to the step 1 material and blended.
3. The blend of step 2 was compressed into mini tablets.

### Stage II: Seal Coating

4. Hydroxypropylmethyl cellulose was dissolved in an isopropyl alcohol:water mixture (95:05) under mechanical stirring until a clear dispersion was formed.
5. The core mini tablets of step 3 were coated with the dispersion of step 4.

### Stage III: Enteric Coating

6. Triethyl citrate and talc were dispersed in purified water using a homogenizer to obtain a dispersion.
7. A simethicone emulsion was added into the dispersion of step 6.
8. The dispersion of step 7 was added into a methacrylic acid copolymer dispersion under continuous stirring, and then filtered.
9. The seal-coated mini tablets of step 5 were coated with the dispersion of step 8 to achieve the desired weight buildup.

### Stage IV: Capsule Filling

10. The enteric-coated mini tablets of step 9 were filled into hard gelatin capsules.

### Pharmacokinetic Studies Under Fasting Conditions

The low dose capsules of Example 14 were compared with Tecfidera® delayed release capsules under fasting conditions in 15 healthy adult human subjects.

Values for various pharmacokinetic parameters, including observed Cₘₐₓ, AUC_{0-∞}, Cₘₐₓ/dose, and AUC_{0-∞}/dose were calculated and are provided in Table 1. Comparative pharmacokinetic data is provided in Table 2.

Reference (R): Tecfidera® capsules 240 mg
Test (T): Dimethyl fumarate capsules 192 mg (Example 14)

TABLE 1
Values for various pharmacokinetic parameters

| | Cₘₐₓ | AUC_{0-inf} | Cₘₐₓ/Dose | AUC_{0-inf}/Dose |
|---|---|---|---|---|
| | (ng/mL) | (ng.h/mL) | (ng/mL/mg) | (ng.h/mL/mg) |
| Reference | 2248.21 | 3939.5223 | 9.37 | 16.41 |
| Example 14 | 2381.65 | 3621.9794 | 12.40 | 18.86 |

TABLE 2
Comparative pharmacokinetic data for low dose capsules of Example
14 (T) and Tecfidera ® (R) in healthy adult human subjects

| | ln Cₘₐₓ | ln AUC₀₋ₜ | ln AUC_{0-inf} |
|---|---|---|---|
| Ratio T/R (%) | 105.93 | 92.33 | 91.93 |
| 90% Confidence | 91.22-123.01 | 86.73-98.30 | 86.43-97.78 |
| Interval | | | |

As is evident from the above data in Table 2, the test product is bioequivalent (same rate and extent of absorption) to the reference product.

### Example 15: Composition Comprising an Enzyme Inhibitor and an Alcohol-Resistant Coat

| Ingredients | Percent (%) w/w |
|---|---|
| Core tablet | |
| Dimethyl fumarate (192 mg | 55.16 |
| or 204 mg) | |
| Microcrystalline cellulose | 5.56 |
| Croscarmellose sodium | 4.60 |
| Sodium lauryl sulfate | 5.94 |
| Disodium EDTA | 1.15 |
| Colloidal silicon dioxide | 0.75 |
| Magnesium stearate | 0.40 |
| Seal coat | |
| Hydroxypropylmethyl cellulose | 2.21 |
| Isopropyl alcohol | q.s. |
| Purified water | q.s. |
| Alcohol-resistant coat | |
| Sodium alginate | 6.11 |
| Polyethylene glycol | 0.90 |
| Talc | 8.14 |
| Purified water | q.s. |
| Enteric coat | |
| Methacrylic acid copolymer | 5.34 |
| dispersion | |
| Triethyl citrate | 1.07 |
| Talc | 2.67 |
| Simethicone | 0.02 |
| Purified water | q.s. |

### Manufacturing Procedure:

### Stage I: Tablet Compression

1. Dimethyl fumarate, microcrystalline cellulose, croscarmellose sodium, sodium lauryl sulfate, disodium EDTA, and colloidal silicon dioxide were blended in a blender.
2. Magnesium stearate was added to the step 1 material and blended.
3. The blend of step 2 was compressed into mini tablets.

### Stage II: Seal Coating

4. Hydroxypropylmethyl cellulose was dissolved in an isopropyl alcohol:water mixture (95:05) under mechanical stirring until a clear dispersion was formed.
5. The core mini tablets of step 3 were coated with the dispersion of step 4.

### Stage III: Alcohol-Resistant Coating

6. Sodium alginate, polyethylene glycol, and talc were dissolved in purified water under mechanical stirring until a dispersion was formed.
7. The seal-coated mini tablets of step 5 were coated with the dispersion of step 6.

### Stage IV: Enteric Coating

8. Triethyl citrate and talc were dispersed in purified water using a homogenizer to obtain a dispersion.
9. A simethicone emulsion was added into the dispersion of step 8.
10. The dispersion of step 9 was added into a methacrylic acid copolymer dispersion under continuous stirring, and then filtered.
11. The coated mini tablets of step 7 were coated with the dispersion of step 10 to achieve the desired weight buildup.

### Stage IV: Capsule Filling

12. The enteric-coated mini tablets of step 11 were filled into hard gelatin capsules.

### Alcohol Dose Dumping Study:

The low-dose capsules of Example 15 (192 mg) were compared with the marketed delayed release Tecfidera® capsules (120 mg) for the release profile in 900 mL of alcoholic media (0.1N HCl with 40% ethanol) using USP apparatus II (paddle) at 100 RPM, and were found to have the following release profile:

TABLE 3
Dimethyl fumarate release in alcoholic media

| Time | Percent Dimethyl Fumarate Release | |
|---|---|---|
| (minutes) | Example 15 | Tecfidera ® |
| 0 | 0 | 0 |
| 15 | 1 | 81.29 |
| 60 | 1 | 98.71 |
| 90 | 1 | 98.65 |
| 120 | 2 | 98.97 |

As is evident from the above data in Table 3, the low-dose capsule of Example 15 is resistant to dose dumping in the presence of alcohol, whereas Tecfidera® is susceptible to dose dumping in the presence of alcohol.

### Pharmacokinetic Studies Under Fasting Conditions

The low-dose capsules of Example 15 (192 mg) was compared with Tecfidera® delayed release capsules (240 mg) under fasting conditions in 15 healthy adult human subjects. Values for various pharmacokinetic parameters, including observed Cₘₐₓ, AUC_{0-∞}, Cₘₐₓ/dose, and AUC_{0-∞}/dose, are provided in Table 4. Comparative pharmacokinetic data is provided in Table 5.

Reference (R): Tecfidera® capsule 240 mg
Test (T): Dimethyl fumarate capsule 192 mg (Example 15)

TABLE 4
Values for various pharmacokinetic parameters

| | Cₘₐₓ | AUC_{0-inf} | Cₘₐₓ/Dose | AUC_{0-inf}/Dose |
|---|---|---|---|---|
| | (ng/mL) | (ng.h/mL) | (ng/mL/mg) | (ng.h/mL/mg) |
| Reference | 2248.21 | 3939.5223 | 9.37 | 16.41 |
| Example 15 | 2873.51 | 3791.7753 | 14.97 | 19.75 |

TABLE 5
Comparative pharmacokinetic data for low-dose capsules of Example
15 (T) and Tecfidera ® (R) in healthy adult human subjects

| | ln Cₘₐₓ | ln AUC₀₋ₜ | ln AUC_{0-inf} |
|---|---|---|---|
| Ratio T/R (%) | 127.81 | 96.68 | 96.24 |
| 90% Confidence | 110.59-147.71 | 91.00-102.72 | 90.67-102.17 |
| Interval | | | |

As is evident from the above data in Table 5, the test product has a higher rate of absorption as compared to the reference product.

### Example 16: Composition Comprising a Permeation Enhancer

| Ingredients | Percent (%) w/w |
|---|---|
| Core tablet | |
| Dimethyl Fumarate (192 mg) | 56.04 |
| Microcrystalline cellulose | 5.65 |
| Croscarmellose sodium | 4.67 |
| Sodium lauryl sulfate | 6.03 |
| Colloidal silicon dioxide | 0.76 |
| Magnesium stearate | 0.40 |
| Seal coat | |
| Hydroxypropylmethyl cellulose | 2.21 |
| Isopropyl alcohol | q.s. |
| Purified water | q.s. |
| Sodium alginate coat | |
| Sodium alginate | 6.11 |
| Polyethylene glycol | 0.90 |
| Talc | 8.14 |
| Purified water | q.s. |
| Enteric coat | |
| Methacrylic acid copolymer dispersion | 5.34 |
| Triethyl citrate | 1.07 |
| Talc | 2.67 |
| Simethicone | 0.02 |
| Purified water | q.s. |

### Manufacturing Procedure:

The capsule of Example 16 is prepared by following the same procedure as for Example 15, with the exception that disodium EDTA is not present in the core.

### Example 17: Composition Comprising an Enzyme Inhibitor and an Alcohol-Resistant Coat

| | |
|---|---|
| Ingredients | Percent (%) w/w |

Core tablet

| | |
|---|---|
| Dimethyl fumarate (180 mg or 90 mg or 96 mg) | 55.16 |
| Microcrystalline cellulose | 5.56 |
| Croscarmellose sodium | 4.60 |
| Sodium lauryl sulfate | 5.94 |
| Disodium EDTA | 1.15 |
| Colloidal silicon dioxide | 0.75 |
| Magnesium stearate | 0.40 |

Seal coat

| | |
|---|---|
| Hydroxypropylmethyl cellulose | 2.21 |
| Isopropyl alcohol | q.s. |
| Purified water | q.s. |

Alcohol-resistant coat

| | |
|---|---|
| Sodium alginate | 6.11 |
| Polyethylene glycol | 0.90 |
| Talc | 8.14 |
| Purified water | q.s. |

Enteric coat

| | |
|---|---|
| Methacrylic acid copolymer dispersion | 5.34 |
| Triethyl citrate | 1.07 |
| Talc | 2.67 |
| Simethicone | 0.02 |
| Purified water | q.s. |

### Manufacturing Procedure:

The capsule of Example 17 is prepared by following the same procedure as for Example 15.

1. An oral pharmaceutical composition comprising dimethyl fumarate; an enzyme modulator or a permeation enhancer or both; and one or more pharmaceutically acceptable excipients.

2. The pharmaceutical composition according to claim 1, wherein the enzyme modulator is an esterase inhibitor or an esterase inducer.

3. The pharmaceutical composition according to claim 2, wherein the esterase inhibitor is selected from the group consisting of EDTA and its salts; sodium lauryl sulfate, Tween® 20, polyoxyl 40 stearate, polyoxyl 35 castor oil, ascorbic acid, lecithin, polyoxyl 40 hydrogenated castor oil, Triton® X-100, poloxamer 188, Tween® 80, PEG 200, PEG 400, PEG 6000, PEG 4000, sodium alginate, mannitol, lactose, and mixtures thereof.

4. The pharmaceutical composition according to claim 2, wherein the esterase inducer is one or more calcium salts.

5. The pharmaceutical composition according to claim 1, wherein the pharmaceutically acceptable excipients are one or more of fillers, binders, disintegrants, lubricants, or glidants.

6. The pharmaceutical composition according to claim 1, wherein the composition further comprises a release modifier.

7. The pharmaceutical composition according to claim 5, wherein the release modifier is a rate-controlling agent.

8. The pharmaceutical composition according to claim 1, wherein the composition is coated with an enteric polymer.

9. The pharmaceutical composition according to claim 1, wherein the composition is coated with an alcohol-resistant polymer.

10. The pharmaceutical composition according to claim 1, wherein the composition is in the form of tablets, capsules, powder, caplets, beads, pellets, pellets in capsules, granules, granules in capsules, minitablets, minitablets in capsules, or sachet.

11. The pharmaceutical composition according to claim 1, wherein the composition is administered one, two, or three times a day.

12. The pharmaceutical composition according to claim 1, wherein the dimethyl fumarate is present in an amount which is at least about 10% lower than the amount of dimethyl fumarate present in the formulation marketed under the trade name Tecfidera®.

13. The pharmaceutical composition according to claim 1, wherein the dimethyl fumarate is present in an amount which is at least about 15% lower than the amount of dimethyl fumarate present in the formulation marketed under the trade name Tecfidera®.

14. The pharmaceutical composition according to claim 1, wherein the dimethyl fumarate is present in an amount which is at least about 20% lower than the amount of dimethyl fumarate present in the formulation marketed under the trade name Tecfidera®.

15. The pharmaceutical composition according to claim 1, wherein the dimethyl fumarate is present in an amount which is at least about 25% lower than the amount of dimethyl fumarate present in the formulation marketed under the trade name Tecfidera®.

16. The pharmaceutical composition according to claim 1, wherein the dimethyl fumarate is present in an amount which is at least about 30% lower than the amount of dimethyl fumarate present in the formulation marketed under the trade name Tecfidera®.

17. The pharmaceutical composition according to claim 1, wherein the dimethyl fumarate is present in an amount which is at least about 35% lower than the amount of dimethyl fumarate present in the formulation marketed under the trade name Tecfidera®.

18. The pharmaceutical composition according to claim 1, wherein the dimethyl fumarate is present in an amount which is at least about 40% lower than the amount of dimethyl fumarate present in the formulation marketed under the trade name Tecfidera®.

19. The pharmaceutical composition according to claim 1, wherein the pharmaceutical composition, when administered orally at a low dose, provides one or both of rate and extent of absorption of dimethyl fumarate equal to or greater than that obtained by a dimethyl fumarate formulation marketed under the trade name Tecfidera®.

20. The pharmaceutical composition according to claim 19, wherein the low dose is about 432 mg per day, about 408 mg per day, about 384 mg per day, about 360 mg per day, about 336 mg per day, about 312 mg per day, or about 288 mg per day.

21. The pharmaceutical composition according to claim 19, wherein the low dose is 432 mg per day, 408 mg per day, 384 mg per day, 360 mg per day, 336 mg per day, 312 mg per day, or 288 mg per day.

22. The pharmaceutical composition according to claim 19, wherein the low dose is 432 mg per day administered as 216 mg capsules twice a day; the low dose is 408 mg per day administered as 204 mg capsules twice a day; the low dose is 384 mg per day administered as 192 mg capsules twice a day; the low dose is 360 mg per day administered as 180 mg capsules twice a day; the low dose is 336 mg per day administered as 168 mg capsules twice a day; the low dose is 312 mg per day administered as 156 mg capsules twice a day; or the low dose is 288 mg per day administered as 144 mg capsules twice a day.

23. The pharmaceutical composition according to claim 9, wherein the pharmaceutical composition is resistant to dose dumping in the presence of alcohol.

24. The pharmaceutical composition according to claim 23, when tested in 900 mL of hydro-alcoholic media (0.1N HCl with 5% to 40% ethanol) in USP dissolution apparatus II, releases not more than 25% of the total drug content after 2 hours.

25. The pharmaceutical composition according to any one of claims 1-22, wherein the pharmaceutical composition, when administered orally to healthy subjects under fasting conditions, provides a mean Cₘₐₓ/dose in the range of about 11.23 to about 16.85 ng/mL/mg.

26. The pharmaceutical composition according to any one of claims 1-22, wherein the pharmaceutical composition, when administered orally to healthy subjects under fasting conditions, provides a mean AUC/dose in the range of about 18.42 to about 27.63 ng·hr/mL/mg.

27. An oral pulsatile-release pharmaceutical composition comprising dimethyl fumarate and one or more pharmaceutically acceptable excipients.

28. The pharmaceutical composition according to claim 27, wherein the composition comprises one or more modified release components or a combination of an immediate release component and a modified release component.

29. The pharmaceutical composition according to claim 28, wherein the modified release component is an extended release component, delayed release component, delayed extended release component, colon-targeted release component, or a combination thereof.

30. The pharmaceutical composition according to claim 27, wherein the composition comprises a delayed release component and a colon-targeted release component.

31. The pharmaceutical composition according to claim 27, wherein the pharmaceutically acceptable excipients are one or more fillers, binders, disintegrants, lubricants, or glidants.

32. The pharmaceutical composition according to claim 27, wherein the composition further comprises an enzyme modulator, a permeation enhancer, or both.

33. The pharmaceutical composition according to claim 27, wherein the composition is coated with an enteric polymer.

34. The pharmaceutical composition according to claim 27, wherein the composition is coated with an alcohol-resistant polymer.

35. The pharmaceutical composition according to claim 27, wherein the composition is in the form of tablets, capsules, powder, caplets, beads, pellets, pellets in capsules, granules, granules in capsules, minitablets, minitablets in capsules, or sachet.

36. The pharmaceutical composition according to claim 27, wherein the composition is administered one, two, or three times a day.

37. The pharmaceutical composition according to claim 27, wherein the dimethyl fumarate is present in an amount which is at least about 10% lower than the amount of dimethyl fumarate present in the formulation marketed under the trade name Tecfidera®.

38. The pharmaceutical composition according to claim 27, wherein the dimethyl fumarate is present in an amount which is at least about 15% lower than the amount of dimethyl fumarate present in the formulation marketed under the trade name Tecfidera®.

39. The pharmaceutical composition according to claim 27, wherein the dimethyl fumarate is present in an amount which is at least about 20% lower than the amount of dimethyl fumarate present in the formulation marketed under the trade name Tecfidera®.

40. The pharmaceutical composition according to claim 27, wherein the dimethyl fumarate is present in an amount which is at least about 25% lower than the amount of dimethyl fumarate present in the formulation marketed under the trade name Tecfidera®.

41. The pharmaceutical composition according to claim 27, wherein the dimethyl fumarate is present in an amount which is at least about 30% lower than the amount of dimethyl fumarate present in the formulation marketed under the trade name Tecfidera®.

42. The pharmaceutical composition according to claim 27, wherein the dimethyl fumarate is present in an amount which is at least about 35% lower than the amount of dimethyl fumarate present in the formulation marketed under the trade name Tecfidera®.

43. The pharmaceutical composition according to claim 27, wherein the dimethyl fumarate is present in an amount which is at least about 40% lower than the amount of dimethyl fumarate present in the formulation marketed under the trade name Tecfidera®.

44. The pharmaceutical composition according to claim 27, wherein the pharmaceutical composition when administered orally at a low dose, provides one or both of rate and extent of absorption of dimethyl fumarate equal to or greater than that obtained by a dimethyl fumarate formulation marketed under the trade name Tecfidera®.

45. The pharmaceutical composition according to claim 44, wherein the low dose is about 432 mg per day, about 408 mg per day, about 384 mg per day, about 360 mg per day, about 336 mg per day, about 312 mg per day, or about 288 mg per day.

46. The pharmaceutical composition according to claim 44, wherein the low dose is 432 mg per day, 408 mg per day, 384 mg per day, 360 mg per day, 336 mg per day, 312 mg per day, or 288 mg per day.

47. The pharmaceutical composition according to claim 44, wherein the low dose is 432 mg per day administered as 216 mg capsules twice a day; the low dose is 408 mg per day administered as 204 mg capsules twice a day; the low dose is 384 mg per day administered as 192 mg capsules twice a day; the low dose is 360 mg per day administered as 180 mg capsules twice a day; the low dose is 336 mg per day administered as 168 mg capsules twice a day; the low dose is 312 mg per day administered as 156 mg capsules twice a day; or the low dose is 288 mg per day administered as 144 mg capsules twice a day.

48. The pharmaceutical composition according to claim 34, wherein the pharmaceutical composition is resistant to dose dumping in the presence of alcohol.

49. The pharmaceutical composition according to claim 48, when tested in 900 mL of hydro-alcoholic media (0.1N HCl with 5% to 40% ethanol) in USP dissolution apparatus II, releases not more than 25% of the total drug content after 2 hours.

50. A method of treating a disease condition requiring dimethyl fumarate therapy, said method comprising orally administering to a subject in need thereof an oral pharmaceutical composition according to claim 1 or claim 27.

51. The method according to claim 50, wherein the disease condition is multiple sclerosis or psoriasis.

52. The method according to claim 50, wherein the disease condition is multiple sclerosis.

53. A method of treating multiple sclerosis, said method comprising orally administering to a subject in need thereof dimethyl fumarate at a low dose of about 288 mg per day to about 384 mg per day.

54. The method according to claim 53, wherein the dimethyl fumarate is administered at a low dose of about 288 mg per day to about 378 mg per day.

55. The method according to claim 53, wherein the dimethyl fumarate is administered at a low dose of about 360 mg per day to about 378 mg per day.

56. The method according to claim 53, wherein the dimethyl fumarate is administered at a low dose of about 360 mg per day.

57. The method according to claim 53, wherein the dimethyl fumarate, when administered at low dose to healthy subjects under fasting conditions, provides a mean Cₘₐₓ/dose in the range of about 11.23 to about 16.85 ng/mL/mg.

58. The method according to claim 53, wherein the dimethyl fumarate, when administered at low dose to healthy subjects under fasting conditions, provides a mean AUC/dose in the range of about 18.42 to about 27.63 ng·hr/mL/mg.
